SEQUENCE LISTING (2 sequences)

SEQ ID NO: 1 - protein, 278 aa, Bos taurus
  misc_feature (152)..(154): /note=“RGD” motif
MRIAVICFCLLGIASALPVKPTSSGSSEEKQLNNKYPDAVATWLKPDPSQKQTFLAPQNSVSSEETDDNKQNTLPSKSNESPEQTDDLDDDDDNSQDVNSNDSDDAETTDDPDHSDESHHSDESDEVDFPTDIPTIAVFTPFIPTESANDGRGDSVAYGLKSRSKKFRRSNVQSPDATEEDFTSHIESEEMHDAPKKTSQLTDHSKETNSSELSKELTPKAKDKNKHSNLIESQENSKLSQEFHSLEDKLDLDHKSEEDKHLKIRISHELDSASSEVN

SEQ ID NO: 2 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

(19) United States
(12) Patent Application Publication
(10) Pub. No.: US 2015/0344534 A1
(43) Pub. Date: Dec. 3, 2015

(54) OSTEOPONTIN VARIANTS FOR USE IN SUPPRESSION OR PREVENTION OF TUMOR GROWTH AND COMPOSITIONS CONTAINING SUCH OSTEOPONTIN VARIANTS

(71) Applicants: ARLA FOODS AMBA, Viby J (DK); FORSYTH DENTAL INFIRMARY FOR CHILDREN (d.b.a. THE FORSYTH INSTITUTE), Cambridge, MA (US)

(72) Inventors: SUSAN R RITTLING, BROOKLINE, MA (US); PETER LANGBORG WEJSE, AARHUS N (DK); ANJA SERENA, MAMMEN, BJERRINGBRO (DK)

(21) Appl. No.: 14/363,565

(22) PCT Filed: Dec. 7, 2012
(86) PCT No.: PCT/US2012/068602

§ 371 Date: Jun. 6, 2014

Related U.S. Application Data
(60) Provisional application No. 61/567,899, filed on Dec. 7, 2011.
(60) Provisional application No. 61/673,912, filed on Jul. 20, 2012.

(30) Foreign Application Priority Data
Jul. 20, 2012 (EP) 12177329.5

(51) Int. Cl. C07K 14/47; A61K 45/06; A23L 1/305; A61K 38/17

(52) U.S. Cl. CPC C07K 14/47; A61K 38/1709; A61K 45/06; A23L 1/305; A23V 2002/00

(57) ABSTRACT

The present invention relates to pharmaceutical compositions and nutritional supplements comprising an osteopontin variant, and medical use of such compositions and supplements for treating or preventing tumor-generating cancer.

### FIELD OF THE INVENTION

The present invention relates to pharmaceutical compositions and nutritional supplements comprising an osteopontin variant, and medical uses of such an OPN variant for treating or preventing tumor-generating cancer.

### BACKGROUND OF THE INVENTION

Osteopontin (OPN) is a secreted, adhesive glycophosphoprotein originally isolated from the collagenous extracellular matrix of mineralized bone (Franzen 1985). OPN is expressed by a number of different cell types, including osteoblasts, arterial smooth muscle cells, leukocytes, several types of epithelial cells, and transformed cells of different lineages (Denhardt 1995). Accordingly, OPN has been detected in many tissues, including kidney, placenta, secretory epithelia and ganglia of the inner ear, and smooth muscle of the vascular system (Butler 1996). OPN is also present in many body fluids, for example plasma, urine, bile and milk, and it displays elevated expression in many transformed cells (Senger 1988). This protein is highly acidic with approximately 25% of the amino acid being aspartate/aspartic acid and glutamate/glutamic acid, and OPN has a significant number of phosphorylated amino acids (Sorensen 1994).

### SUMMARY OF THE INVENTION

The present inventors have made the surprising discovery that orally administered osteopontin variants suppress, and possibly even prevent, the growth of cancer tumors. This effect was completely unexpected. First of all, administration of OPN has never been documented to have beneficial effects relating to the treatment of cancer, and secondly, it is very surprising that an orally administered proteins and peptides have medical effects outside the gastrointestinal system.

Thus, an aspect of the invention pertains to an OPN variant for use in the treatment or prevention of cancer involving at least one cancer tumor.

For example, an aspect of the invention pertains to an OPN variant for use in the treatment or prevention of cancer involving at least one cancer tumor, wherein said OPN variant comprises an active OPN molecule having a sequence identity of at least 80% relative to SEQ ID NO. 1 or SEQ ID NO. 2, and/or an active fragment of an OPN molecule, said fragment having a sequence identity of at least 80% relative to the sequence of position 17-163 of SEQ ID NO. 1 or to the sequence of position 17-170 of SEQ ID NO. 2.

In the context of the present invention, the term “cancer involving a cancer tumor” pertains to cancer diseases during which at least one cancer tumor is formed in or on the patient. The at least one cancer tumor may be the primary cancer tumor of the cancer or a subsequent metastasis.

A further aspect of the invention pertains to a method of treating or preventing cancer, the method comprising: administering to a subject having cancer, or to a subject being at risk of getting cancer, an amount of an OPN variant effective to treat or prevent said cancer, and wherein said cancer involves at least one cancer tumor.

Another aspect of the invention pertains to a pharmaceutical composition comprising:
an OPN variant, and
a pharmaceutically acceptable carrier.

An additional aspect of the invention pertains to a nutritional supplement comprising
a nutritionally effective amount of an OPN variant, and
one or more components selected from the group consisting of a carbohydrate source, a lipid source, and a protein source.

Some aspects provided here relate to methods, comprising administering to a subject having a tumor cell mass an amount of the OPN variant effective to suppress tumor cell growth or replication.

In certain embodiments, the OPN variant is administered at a concentration of about 0.05 mg/ml to about 1 g/ml. In other embodiments, the OPN variant is administered in an amount of about 0.05 mg/kg of body weight to about 5 g/kg.

In particular embodiments, the OPN variant is administered mucosally. In other embodiments, the OPN variant is administered orally, sublingually, buccally, or nasally.

In some embodiments, the tumor cell may be a subcutaneous tumor cell, and in any one of the foregoing embodiments, the tumor cell may reside in a breast, cervix, ovary, prostate, lung, colon, rectum, pancreas, stomach, kidney, or thyroid.

In certain embodiments, the OPN variant is isolated from bovine milk. The OPN variant may e.g. be bovine milk OPN.

In particular embodiments, the OPN variant is recombinant OPN.

In some embodiments, the OPN variant is provided in a purified source of OPN variant.

In certain embodiments, the purified source providing the OPN variant is at least about 95% pure.

Other aspects provided herein are directed to pharmaceutical compositions that include the OPN variant, preferably in an amount of the OPN variant effective to suppress tumor cell growth or replication, and a pharmaceutically acceptable carrier.

In certain embodiments, the amount of the OPN variant in the pharmaceutical composition is about 0.05 mg/ml to about 1 g/ml.

In particular embodiments, the pharmaceutical composition is formulated for mucosal administration. The pharmaceutical composition may e.g. be formulated for oral, sublingual, buccal, or nasal administration.

In some embodiments, the pharmaceutically acceptable carrier is selected from the group consisting of ethanol, glycerine, propylene glycol, polyethylene glycol, sugar solution, sorbitol, buffer, saline, and water.

In certain embodiments, the pharmaceutical composition is a solid, a liquid, or an emulsion.

In particular embodiments, the pharmaceutical composition is administered as a tablet or a spray.

In any one of the foregoing embodiments, the pharmaceutical composition may comprise a taste-masking agent.

In any one of the foregoing embodiments, the pharmaceutical composition may comprise one or more anticancer targeted therapeutic or chemotherapeutic agents.

In any one of the foregoing embodiments, the pharmaceutical composition may comprise one or more immunosuppressive or immunostimulating agents.

Other aspects provided herein are directed to nutritional supplements that include the OPN variant, preferably, in an amount of the OPN variant effective to suppress tumor cell growth or replication.

In some embodiments, the supplement is a liquid or a powder.

In certain embodiments, the supplement comprises one or more fruit(s), one or more vegetable(s), yogurt, milk, ice cream, or a combination thereof.

In any one of the foregoing embodiments, the supplement may be fortified with protein, vitamins, minerals, antioxidants, prebiotics, probiotics, or a combination thereof.

In any one of the foregoing embodiments, the supplement may be lactose-free and/or gluten-free.

In certain embodiments, the supplement is organic.

In some embodiments, the supplement is a smoothie or a juice, while in other embodiments, the supplement is a milk.

These and other aspects of the invention will be described in connection with the drawings and the detailed description below.

### BRIEF DESCRIPTION OF THE DRAWINGS

FIG. 1A shows the tumor volume (cm³, +/−SEM) in mice that received oral doses of OPN preparation at various concentrations, starting on day 0.

FIG. 1B shows the tumor volume (cm³, +/−SEM) in mice that received oral doses of OPN preparation at various concentrations, starting on day 5.

FIGS. 2A-C show a comparison of tumor sizes in all groups on days 15, 17, and 19; significant differences are indicated.

FIG. 3 shows the mean tumor size of control and OPN-fed mice (0.3 mg/ml in drinking water), combined results from three independent experiments. N=30 (0 mg/ml OPN preparation) or 32 (0.3 mg/ml OPN preparation).

### DETAILED DESCRIPTION OF THE INVENTION

As said, an aspect of the invention pertains to an OPN variant for use in the treatment or prevention of cancer involving at least one cancer tumor.

In some preferred embodiments of the invention, the OPN variant comprises, or even consists of, an active OPN molecule having a sequence identity of at least 80% relative to SEQ ID NO. 1 or SEQ ID NO. 2, and/or an active fragment of an OPN molecule, said fragment having a sequence identity of at least 80% relative to the sequence of position 17-163 of SEQ ID NO. 1 or to the sequence of position 17-170 of SEQ ID NO. 2.

In the context of the present invention the term “osteopontin variant” or “OPN variant” pertains to both native OPN which e.g. may be found in mammal milk as well as artificial OPN which contain minor amino acid sequence variations relative to the sequence of native OPN. The OPN variant may e.g. be prepared by purification from natural sources of OPN such as mammal milk, and preferably bovine milk, or it may be prepared by fermentation or synthesis. The term “osteopontin variant” or “OPN variant” pertains to the components of a composition, e.g. a pharmaceutical or nutraceutical supplement which are OPN molecules and peptides derived from OPN molecules, e.g. by hydrolysis or truncation of OPN molecules.

In the context of the present invention the term “osteopontin molecule” or “OPN molecule” pertains to a population of an OPN molecule with a given amino acid sequence. Such a population may contain several phospho-isoforms and glyco-isoforms of the OPN molecule.

The OPN variant may furthermore contain a second active OPN molecule, and even further active OPN molecules.

As will be understood, the OPN variant is a pharmaceutically active OPN variant and contains one or more pharmaceutically active OPN molecule(s) and/or one or more pharmaceutically active fragment(s) of OPN molecules. In the context of the present invention, an OPN variant, an active OPN molecule, or a fragment of an OPN molecule is deemed “pharmaceutically active” or “active” if it is capable of reducing the growth of a cancer tumor in a mammal subject. The pharmaceutical activity of an OPN variant, or the individual OPN molecules or fragments of OPN molecules, may e.g. be tested using the procedure outlined in Example 1.

In some preferred embodiments of the invention, the OPN molecule is a peptide having a sequence identity of at least 80% relative to SEQ ID NO. 1 (Bovine OPN; UniProtKB/Swiss-Prot Entry P31096). For example, the active OPN molecule may be a peptide having a sequence identity of at least 90% relative to SEQ ID NO. 1. Preferably, the active OPN molecule is a peptide having a sequence identity of at least 95% relative to SEQ ID NO. 1. Even more preferably, the active OPN molecule may for example be a peptide having a sequence identity of at least 98% relative to SEQ ID NO. 1. The active OPN molecule may for example have the amino acid sequence of SEQ ID NO. 1.

In the context of the present invention, the term “sequence identity” relates to a quantitative measure of the degree of identity between two amino acid sequences or between two nucleic acid sequences, preferably of equal length. If the two sequences to be compared are not of equal length, they must be aligned to the best possible fit. The sequence identity can be calculated as

(N_{ref}−N_{dif})*100)/(N_{ref}),

wherein N_{dif} is the total number of non-identical residues in the two sequences when aligned, and wherein N_{ref} is the number of residues of the reference sequences. Hence, the DNA sequence AGTCAGTC will have a sequence identity of 75% with the sequence AATCAATC (N_{dif}=2 and N_{ref}=8). A gap is counted as non-identity of the specific residue(s), i.e. the DNA sequence AGTGTC will have a sequence identity of 75% with the DNA sequence AGTCAGTC (Ndif=2 and Nref=8). Sequence identity can for example be calculated using appropriate BLAST-programs, such as the BLASTp-algorithm provided by National Center for Biotechnology Information (NCBI), USA.

In some preferred embodiments of the invention, the active OPN molecule is a peptide having a sequence identity of at least 80% relative to SEQ ID NO. 2 (Human OPN; UniProtKB/Swiss-Prot Entry P10451). For example, the active OPN molecule may be a peptide having a sequence identity of at least 90% relative to SEQ ID NO. 2. Preferably, the active OPN molecule is a peptide having a sequence identity of at least 95% relative to SEQ ID NO. 2. Even more preferably, the active OPN molecule may be a peptide having a sequence identity of at least 98% relative to SEQ ID NO. 2.

In other preferred embodiments of the invention, the active OPN molecule is human OPN (SEQ ID NO. 2).

Alternatively, or additionally, the OPN variant may contain an active fragment of an OPN molecule.

In the context of the present invention the term “active fragment of an osteopontin molecule” or “active fragment of an OPN molecule” pertains to a population of the active fragment of an OPN molecule, said fragment having a given amino acid sequence. Such a population may also contain several phospho-isoforms and glyco-isoforms of the active fragment of the OPN molecule. The “active fragment of an OPN molecule” are preferably native long n-terminal fragments of OPN molecules which e.g. may be found in mammal milk as well as artificial long n-terminal fragments of OPN molecules which contain minor amino acid sequence variations relative to the sequence of native long n-terminal fragment of OPN molecules. The active fragments of OPN molecules may e.g. be prepared by purification from natural sources of OPN fragments such as mammal milk, and preferably bovine milk, or it may be prepared by fermentation or synthesis.

Thus, in some preferred embodiments of the invention, the OPN variant contains a first population of an active OPN molecule and a second population of an active fragment of an OPN molecule. Again, these populations may contain several phospho-isoforms and glyco-isoforms of the active OPN molecule and of the active fragment of an OPN molecule.

In some preferred embodiments of the invention, the active fragment of an OPN molecule is a peptide having a sequence identity of at least 80% relative to the sequence of position 17-163 of SEQ ID NO. 1 (position 17-163 of Bovine OPN; UniProtKB/Swiss-Prot Entry P31096). For example, the active fragment of an OPN molecule may be a peptide having a sequence identity of at least 90% relative to the sequence of position 17-163 of SEQ ID NO. 1. Preferably, the active fragment of an OPN molecule is a peptide having a sequence identity of at least 95% relative to the sequence of position 17-163 of SEQ ID NO. 1. Even more preferably, the active fragment of an OPN molecule may for be a peptide having a sequence identity of at least 98% relative to the sequence of position 17-163 of SEQ ID NO. 1.

In some preferred embodiments of the invention, the active fragment of an OPN molecule is a peptide having a sequence identity of at least 80% relative to the sequence of position 17-170 of SEQ ID NO. 2 (position 17-170 of Human OPN; UniProtKB/Swiss-Prot Entry P10451). For example, the active fragment of an OPN molecule may be a peptide having a sequence identity of at least 90% relative to the sequence of position 17-170 of SEQ ID NO. 2. Preferably, the active fragment of an OPN molecule is a peptide having a sequence identity of at least 95% relative to the sequence of position 17-170 of SEQ ID NO. 2. Even more preferably, the active fragment of an OPN molecule may be a peptide having a sequence identity of at least 98% relative to the sequence of position 17-170 of SEQ ID NO. 2.

In some preferred embodiments of the invention, the OPN variant is native OPN, i.e. an OPN variant which is naturally occurring in a mammal, e.g. in mammal milk.

In some embodiments of the invention, the OPN variant comprises a total amount of active fragments of OPN molecules of at least 10% (w/w) relative to the total weight of the OPN variant. The OPN variant may for example comprise a total amount of active fragments of OPN molecules of at least 15% (w/w) relative to the total weight of the OPN variant. Alternatively, the OPN variant may comprise a total amount of active fragments of OPN molecules of at least 30% (w/w) relative to the total weight of the OPN variant. The OPN variant may e.g. comprise a total amount of active fragments of OPN molecules of at least 50% (w/w) relative to the total weight of the OPN variant.

Even higher contents of the active fragments may be desired, thus, in some embodiments of the invention, the OPN variant comprises a total amount of active fragments of OPN molecules of at least 60% (w/w) relative to the total weight of the OPN variant. The OPN variant may for example comprise a total amount of active fragments of OPN molecules of at least 70% (w/w) relative to the total weight of the OPN variant. Alternatively, the OPN variant may comprise a total amount of active fragments of OPN molecules of at least 80% (w/w) relative to the total weight of the OPN variant. The OPN variant may e.g. comprise a total amount of active fragments of OPN molecules of at least 90% (w/w) relative to the total weight of the OPN variant. For example, the OPN variant may comprise a total amount of active fragments of OPN molecules of at least 95% (w/w) relative to the total weight of the OPN variant.

In some embodiments of the invention, the OPN variant comprises a total amount of active OPN molecules in the range of 10-90% (w/w) relative to the total weight of the OPN variant, and a total amount of active fragments of OPN molecules in the range of 10-90% (w/w) relative to the total weight of the OPN variant.

In some embodiments of the invention, the OPN variant comprises a total amount of active OPN molecules in the range of 10-40% (w/w) relative to the total weight of the OPN variant, and a total amount of active fragments of OPN molecules in the range of 60-90% (w/w) relative to the total weight of the OPN variant.

For example, the OPN variant may comprise a total amount of active OPN molecules in the range of 15-35% (w/w) relative to the total weight of the OPN variant, and a total amount of active fragments of OPN molecules in the range of 75-85% (w/w) relative to the total weight of the OPN variant.

In some embodiments of the invention, the OPN variant comprising a total amount of active OPN molecules of at least 10% (w/w) relative to the total weight of the OPN variant.

The OPN variant may for example comprise a total amount of active OPN molecules of at least 15% (w/w) relative to the total weight of the OPN variant. Alternatively, the OPN variant may comprise a total amount of active OPN molecules of at least 30% (w/w) relative to the total weight of the OPN variant. The OPN variant may e.g. comprise a total amount of active OPN molecules of at least 50% (w/w) relative to the total weight of the OPN variant.

Even higher contents of the active fragments may be desired, thus, in some embodiments of the invention, the OPN variant comprises a total amount of active OPN molecules of at least 60% (w/w) relative to the total weight of the OPN variant. The OPN variant may for example comprise a total amount of active OPN molecules of at least 70% (w/w) relative to the total weight of the OPN variant. Alternatively, the OPN variant may comprise a total amount of active OPN molecules of at least 80% (w/w) relative to the total weight of the OPN variant. The OPN variant may e.g. comprise a total amount of active OPN molecules of at least 90% (w/w) relative to the total weight of the OPN variant. For example, the OPN variant may comprise a total amount of active OPN molecules of at least 95% (w/w) relative to the total weight of the OPN variant.

In some embodiments of the invention, the OPN variant is isolated from bovine milk.

In some preferred embodiments of the invention, the OPN variant is bovine OPN. Bovine OPN may for example be isolated from bovine milk, in which case it normally both phosphorylated and glycosylated. It is also possible to use OPN from bovine milk in dephosphorylated and/or de-glycosylated form.

Bovine milk OPN contains both bovine full length OPN (SEQ ID NO. 1) and a truncated OPN isoform (a peptide having the sequence of position 17-163 of SEQ ID NO. 1).

Thus, in some preferred embodiments of the invention, the OPN variant comprises, or even consists of, an active OPN molecule having a sequence identity of at least 80% relative to SEQ ID NO. 1, and an active fragment of an OPN molecule having a sequence identity of at least 80% relative to the sequence of position 17-163 of SEQ ID NO. 1.

For example, the OPN variant may comprise, or even consist of, an active OPN molecule having a sequence identity of at least 90% relative to SEQ ID NO. 1, and an active fragment of an OPN molecule having a sequence identity of at least 90% relative to the sequence of position 17-163 of SEQ ID NO. 1. Alternatively, the OPN variant may comprise, or even consist of, an active OPN molecule having a sequence identity of at least 95% relative to SEQ ID NO. 1, and an active fragment of an OPN molecule having a sequence identity of at least 95% relative to the sequence of position 17-163 of SEQ ID NO. 1. For example, OPN variant may comprise, or even consist of, an active OPN molecule having the sequence of SEQ ID NO. 1, and an active fragment of an OPN molecule having the sequence of position 17-163 of SEQ ID NO. 1.

In some embodiments of the invention, the OPN variant may comprise, or even consist of, an active OPN molecule having a sequence identity of at least 80% relative to SEQ ID NO. 2, and/or an active fragment of an OPN molecule having a sequence identity of at least 80% relative to the sequence of position 17-170 of SEQ ID NO. 2.

For example, the OPN variant may comprise, or even consist of, an active OPN molecule having a sequence identity of at least 90% relative to SEQ ID NO. 2, and an active fragment of an OPN molecule having a sequence identity of at least 90% relative to the sequence of position 17-170 of SEQ ID NO. 2. Alternatively, the OPN variant may comprise, or even consist of, an active OPN molecule having a sequence identity of at least 95% relative to SEQ ID NO. 2, and an active fragment of an OPN molecule having a sequence identity of at least 95% relative to the sequence of position 17-170 of SEQ ID NO. 2. For example, OPN variant may comprise, or even consist of, an active OPN molecule having the sequence of SEQ ID NO. 2, and an active fragment of an OPN molecule having the sequence of position 17-170 of SEQ ID NO. 2.

A cancer tumor comprises several tumor cells (neoplastic cells), which are characterized by abnormal cell growth or replication. In some instances, abnormal cell growth (for example of a localized region of cells) results in formation of a tumor cell mass (neoplasm, solid lesion). Abnormal cell growth is relative to the growth of cells which do not form a tumor cell mass. Abnormal cells may exhibit an abnormal (for example increased) rate of division as compared to normal cells. In some embodiments, tumor cells are pre-malignant or malignant. Malignant tumor cells may be referred to as cancer cells and may have the ability to metastasize or spread to neighboring tissues or other locations in the body and grow there as new tumors.

It is particularly preferred that the at least one cancer tumor has an elevated level of OPN.

Thus, in some preferred embodiments of the invention the OPN variant is for use in the treatment or prevention of cancer involving at least one cancer tumor, which cancer tumor has an elevated level of OPN.

In additional preferred embodiments of the invention the tumor is capable of inducing an elevated concentration of OPN in the plasma of the subject having the tumor.

In certain embodiments, the tumor cells are of epithelial origin. Epithelial cells reside in one or more layers which cover the entire surface of the body and which line most of the hollow structures of the body, excluding the blood vessels, lymph vessels, and the heart interior, which are lined with endothelium, and the chest and abdominal cavities which are lined with mesothelium.

Epithelial tumors include benign and premalignant epithelial tumors, such as breast fibroadenoma and colon adenoma, and malignant epithelial tumors. Malignant epithelial tumors include primary tumors, also referred to as carcinomas, and secondary tumors, also referred to as metastases of epithelial origin. Carcinomas include acinar carcinoma, acinous carcinoma, alveolar adenocarcinoma (also called adenocystic carcinoma, adenomyoepithelioma, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliale adenoides, carcinoma exulcere, carcinoma fibrosum, gelatiniform carcinoma, gelatinous carcinoma, giant cell carcinoma, gigantocellulare, glandular carcinoma, granulosa cell carcinoma, hair-matrix carcinoma, hematoid carcinoma, hepatocellular carcinoma (also called hepatoma, malignant hepatoma and hepatocarcinoma), Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypernephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma vilosum.

In some preferred embodiments of the invention, the cancer tumor is a fibrosarcoma.

In other embodiments, the tumor cells are of mesenchymal origin, for example, tumor cells forming a sarcoma. Sarcomas are rare mesenchymal neoplasms that arise in bone and soft tissues. Different types of sarcomas include liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal [not bone] Ewing's sarcoma, and primitive neuroectodermal tumor [PNET]), synovial sarcoma, angiosarcomas, hemangiosarcomas, lymphangiosarcomas, Kaposi's sarcoma, hemangioendothelioma, fibrosarcoma, desmoid tumor (also called aggressive fibromatosis), dermatofibrosarcoma protuberans (DFSP), malignant fibrous histiocytoma (MFH), hemangiopericytoma, malignant mesenchymoma, alveolar softpart sarcoma, epithelioid sarcoma, clear cell sarcoma, desmoplastic small cell tumor, gastrointestinal stromal tumor (GIST) (also known as GI stromal sarcoma), osteosarcoma (also known as osteogenic sarcoma)-skeletal and extraskeletal, and chondrosarcoma.

In some preferred embodiments of the invention, the cancer tumor is an adenocarcinoma or a breast carcinoma.

In some embodiments, the tumor cells are of melanocytic origin. Melanomas are tumors arising from the melanocytic system of the skin and other organs. Examples of melanoma include lentigo maligna melanoma, superficial spreading melanoma, nodular melanoma, and acral lentiginous melanoma.

In still other embodiments, the tumor cells include those found in biliary tract cancer, endometrial cancer, esophageal cancer, gastric cancer, intraepithelial neoplasms, including Bowen's disease and Paget's disease, liver cancer, oral cancer, including squamous cell carcinoma, sarcomas, including fibrosarcoma and osteosarcoma, skin cancer, including melanoma, Kaposi's sarcoma, testicular cancer, including germinal tumors (seminoma, non-seminoma (teratomas, choriocarcinomas)), stromal tumors and germ cell tumors, thyroid cancer, including thyroid adenocarcinoma and medullar carcinoma, and renal cancer, including adenocarcinoma and Wilms' tumor.

In particular embodiments, the tumor cells may originate in bone, muscle or connective tissue. The tumor cells may be found in primary tumors (for example sarcomas) of bone and connective tissue.

In other embodiments, the tumor cells may be metastatic. In some embodiments, the metastatic tumors are of epithelial origin. Carcinomas may metastasize to bone, as has been observed with breast cancer, and liver, as is sometimes the case with colon cancer. The methods provided herein are directed to suppressing growth or replication of metastatic tumors irrespective of the site of the metastasis and/or the site of the primary tumor.

The OPN variant may for example comprise an OPN molecule. In some preferred embodiments of the invention, the OPN variant may comprise, or even consist of, an OPN molecule.

The OPN variant, or the individual active OPN molecules or active fragments of OPN molecules may be isolated from natural source of such compounds. Alternatively, the OPN variant may be prepared by fermentation or by chemical synthesis.

In certain embodiments, the OPN variant is isolated from milk, and in particular embodiments, the milk is bovine milk. In other embodiments, the OPN variant is isolated from other domesticated milk producing mammals, including goat, sheep, buffalo, llama, and camel. For methods of isolating OPN variants from milk, see for example, U.S. Pat. No. 7,259,243, the entire disclosure of which is herein incorporated by reference.

In some embodiments, the source of OPN variant is purified with respect to the OPN variant. In some embodiments, the source of OPN variant is at least about 50% to about 60%, at least about 60% to about 70%, or at least about 70% to about 80% pure. In some embodiments, it is at least about 80% to about 90% pure, while in other embodiments, the source of OPN variant is at least about 90% to about 95% pure, or more. In certain embodiments, the purified source of OPN variant is at least about 95% pure, such as 95%, 96%, 97%, 98%, 99%, or 99.5% pure, or more.

In specific embodiments, the purified source of the OPN variant is a purified bovine OPN preparation, such as for example Lacprodan OPN-10® (Arta Foods Ingredients, Viby, Denmark) (See also U.S. Pat. No. 7,259,243). Lacprodan OPN-10® comprises approx. 22% (w/w) full length bovine milk OPN and approx. 65% (w/w) of a bovine milk OPN isoform (a truncated version of full length OPN).

In other embodiments, the OPN variant is a recombinant protein or peptide.

The OPN variant may be administered in several ways.

In some preferred embodiments of the invention the treatment or prevention is by oral administration. Oral administration may for example involve sublingual administration and/or buccal administration. Alternatively, or additionally, oral administration may involve that the OPN variant enters the gastrointestinal system.

Alternatively, the OPN variant may be administered by parenterally, e.g. by injection or infusion. Thus, in some preferred embodiments of the invention the treatment or prevention may for example be by intravenous (IV) administration of the OPN variant. In other preferred embodiments of the invention the treatment or prevention may for example be by intramuscular or subcutaneous administration, such as intramuscular or subcutaneous injection. In other preferred embodiments of the invention the treatment or prevention may for example be by intra-peritoneal administration, such as intra-peritoneal injection.

In further embodiments of the invention the treatment or prevention may for example be by nasal administration.

In some embodiments of the invention the treatment or prevention is for suppressing, and/or reducing, tumor cell growth or replication. For example, the treatment or prevention may be for preventing tumor cell replication. The treatment or prevention may e.g. be for preventing tumor cell growth.

In some preferred embodiments of the invention medical uses and methods provided herein are directed to suppressing, and/or reducing, growth or replication of tumor cells regardless of their site of origin.

The treatment or prevention may also be for suppressing, and/or reducing, cancer tumor growth.

In some preferred embodiments of the invention, the treatment is for preventing growth of a cancer tumor. For example, the treatment may be for preventing growth and/or replication of the cancer cells of a cancer tumor.

In some embodiments of the invention the treatment or prevention is for reducing the risk of metastasis in a subject having a cancer involving at least one cancer tumor. For example, the treatment or prevention may be for preventing metastasis in a subject having a cancer involving at least one cancer tumor.

The treatment or prevention may for example prevent tumor cell growth or replication.

In some preferred embodiments of the invention, the subject to be treated is a human subject.

In some preferred embodiments of the invention, the subject to be treated has a cancer involving at least one cancer tumor having an elevated level of OPN.

In the context of the present invention, a cancer tumor has an elevated level of OPN if the level of OPN in the cancer tumor is at least 1 nanogram/microgram protein. The level of OPN in a cancer tumor is determined according to Example 3. In some embodiments of the invention a cancer tumor is deemed to have an elevated level of OPN if the level of OPN in the cancer tumor is at least 5 nanogram/microgram protein. For example, a cancer tumor may be deemed to have an elevated level of OPN if the level of OPN in the cancer tumor is at least 10 nanogram/microgram protein. In other embodiments of the invention a cancer tumor is deemed to have an elevated level of OPN if the level of OPN in the cancer tumor is at least 20 nanogram/microgram protein. For example, a cancer tumor may be deemed to have an elevated level of OPN if the level of OPN in the cancer tumor is at least 50 nanogram/microgram protein.

Alternatively, an elevated level of OPN in a cancer tumor can be determined by immunohistochemistry essentially as described for human tumor samples (Tuck 1998). 4-6 micron sections of formalin fixed, paraffin embedded tumor tissues are rehydrated and subjected to antigen retrieval by boiling for 12 minutes in 0.01 M NaCitrate, pH 6.0. After blocking in 5% goat serum, anti-osteopontin antibody (R&D # AF808 or Santa Cruz Biotechnologies mAK2A1) will be diluted according to the manufacturer's instructions and incubated with the tissue sections for 1 hour. Secondary antibody incubation and detection is performed using the Vector ABC Elite kit comprising biotinylated antigoat antibody and avidin-biotin complex (Vector cat #PK-6105); detection is achieved by staining with diaminobenzidine (DAB, included in kit). Extent and intensity of staining is determined microscopically and graded according to a semiquantitative system described in (Tuck 1998). Tumor samples with a score greater than 4 will be considered having an elevated level of OPN.

In others preferred embodiments of the invention the subject having the cancer tumor has an elevated concentration of OPN in the plasma derived from its blood.

In the context of the present invention, a subject has an elevated concentration of OPN in its plasma if the plasma concentration of OPN is at least 80 nanogram/mL. The concentration of OPN in plasma derived from the blood of a subject is determined according to Example 4. In some embodiments of the invention a subject has an elevated concentration of OPN in its plasma if the plasma concentration of OPN is at least 100 nanogram/mL. For example, a subject may have an elevated concentration of OPN in its plasma if the plasma concentration of OPN is at least 120 nanogram/mL. In other embodiments of the invention a subject has an elevated concentration of OPN in its plasma if the plasma concentration of OPN is at least 140 nanogram/mL. For example, a subject may have an elevated concentration of OPN in its plasma if the plasma concentration of OPN is at least 180 nanogram/mL.

It should be noted that the OPN measured in the cancer tumor or in plasma is OPN produced by the subject and it need not be identical to the OPN variant.

In some preferred embodiments of the invention, the subject to which the OPN variant is, or is to be, administered has an increased risk of developing a cancer involving at least one cancer tumor.

In the context of the present invention, a subject is deemed to have an increased risk of developing a cancer involving at least one cancer tumor if the subject's lifetime risk of developing a cancer tumor is at least 20% higher than that calculated for persons from the general population matched for gender, age and ethnicity.

An example of increased risk is a 55 year old woman with a first degree relative with breast cancer: this woman's lifetime risk of developing breast cancer is 36% higher than the general population (see e.g. the Breast Cancer Risk Assessment Tool on www.cancer.gov/bcrisktool/ which is an interactive tool designed by scientists at the National Cancer Institute (NCI)).

The increased risk may be cause by hereditary or environmental circumstances or by the life style of the subject.

In some embodiments of the invention, the increased risk is caused by an environmental circumstance. The subject may for example have been exposed to an significant amount of radioactive radiation or to a significant amount of a carcinogenic substance.

In other embodiments of the invention, the increased risk is caused by the life style of the subject. The subject may for example be a tobacco-smoker, or an ex-tobacco-smoker.

In still other embodiments of the invention, the increased risk is caused by heritage from a parent of the subject. The subject may for example have at least one first-degree relative, e.g. a mother, father, sister or brother, which has breast cancer, lung cancer, ovary cancer or colon cancer.

The subject having an increased risk of developing cancer may for example have a genetic profile which is associated with an increased risk of developing a cancer involving at least one cancer tumor.

In the context of the present invention, the term “genetic profile” relates both to the genes which the subject has inherited from its parents and to gene mutations which have been caused by environmental circumstances.

In some embodiments of the invention, the genetic profile comprises at least one inherited gene which is associated with an increased risk of developing a cancer involving at least one cancer tumor. An example of such a gene is the BRCA1 gene or the BRCA2 gene. See for example Nelson 2005.

In the context of the present invention, a genetic profile is deemed to be associated with an increased risk of developing a cancer involving at least one cancer tumor if the lifetime risk of developing the cancer is at least 20% higher for carriers of the genetic profile than for non-carriers.

In some preferred embodiments of the invention, the subject which is, or is to be, treated is also treated with another type of anti-cancer treatment. Examples of such other kinds of anti-cancer treatment are for example chemotherapy, chemoprevention, targeted therapy, bone-marrow transplant, radiation treatment, surgery, or a combination thereof.

In some preferred embodiments of the invention the OPN variant is administered in a daily dosage in the range of about 0.05 mg/kg of body weight to about 5 g/kg of body weight of the subject treated.

The rate of tumor cell growth and, consequently, the overall size of a tumor cell mass may be reduced and in certain embodiments statistically significantly reduced in a subject, if the OPN variant is administered (for example orally) to the subject. Suppression of tumor cell growth resulting from a subject having received an effective amount of the OPN variant is relative to the rate of tumor cell growth of that same tumor prior to the subject receiving the OPN variant, or relative to tumor cell growth of a comparable tumor (comparable in initial size of the mass and cell type) in a subject having not been exposed to an effective amount of the OPN variant. Tumor cell growth may refer to the rate of cell division or replication, or the overall size (for example volume or circumference) of the tumor cell mass. Methods of measuring a tumor cell mass are well known in the art. For example, see Tomayko 1989, incorporated herein by reference.

In the context of the present invention the size of a cancer tumor refers to the volume of the tumor. The rate of growth of a cancer tumor refers to the volumetric growth of the tumor per time unit. The volume of a cancer tumor may be determined by conventional imaging techniques such as MRI scanner or ultrasonic imaging.

In certain embodiments, the rate of growth or size of a tumor cell mass may be reduced by at least about 5% to about 10%, as compared to the rate of growth or size, of a similar tumor not exposed to a therapeutically effective amount of the OPN variant. In other embodiments, the tumor cell mass may be reduced by at least about 10% to about 15%, at least about 15% to about 20%, at least about 20% to about 25%, at least about 25% to about 30%, at least about 30% to about 35%, at least about 35% to about 40%, at least about 40% to about 45%, at least about 45% to about 50%, at least about 50% to about 55%, at least about 55% to about 60%, at least about 60% to about 65%, at least about 65% to about 70%, at least about 70% to about 75%, at least about 75% to about 80%, at least about 80% to about 85%, or at least about 85% to about 90%, or more. In yet other embodiments, the tumor cell mass is reduced by at least about 50%. In particular embodiments, the tumor cell mass is reduced by at least about 75%.

Alternatively, the rate of growth of a tumor cell mass may be reduced about 5% to about 100%, as compared to the rate of growth or size of a similar tumor not exposed to a therapeutically effective amount of the OPN variant. For example, the rate of growth of a tumor cell mass may be reduced about 20% to about 95%. Preferably, the rate of growth of a tumor cell mass is reduced about 40% to about 100%. Even more preferably, the rate of growth of a tumor cell mass is reduced about 60% to about 100%.

The OPN variant, as described herein, may be administered to a subject in an amount effective to suppress growth or replication of tumor cells. In certain embodiments, the OPN variant may be administered at a concentration of about 0.05 ring/ml to about 1 mg/ml. In some embodiments, it may be administered at a concentration of about 0.05 mg/ml to about 0.1 mg/ml, about 0.1 mg/ml to about 0.15 mg/ml, about 0.15 mg/ml to about 0.2 mg/ml, about 0.25 mg/ml to about 0.3 mg/ml, about 0.3 mg/ml to about 0.35 mg/ml, about 0.35 mg/ml to about 0.4 mg/ml, about 0.4 mg/ml to about 0.45 mg/ml, about 0.45 mg/ml to about 0.5 mg/ml, about 0.55 mg/ml to about 0.6 mg/ml, about 0.6 mg/ml to about 0.65 mg/ml, about 0.65 mg/ml to about 0.7 mg/ml, about 0.7 mg/ml to about 0.75 mg/ml, about 0.75 mg/ml to about 0.8 mg/ml, about 0.85 mg/ml to about 0.9 mg/ml, about 0.9 mg/ml to about 0.95 mg/ml, or about 0.95 mg/ml to about 1 mg/ml. In one embodiment, the OPN variant may be administered at a concentration of 0.03 mg/ml. In another embodiment, the OPN variant may be administered at a concentration of 0.12 mg/ml. In yet another embodiment, the OPN variant may be administered at a concentration of 0.3 mg/mi.

In particular embodiments, the OPN variant may be administered at a concentration of about 1 mg/ml to about 0.1 g/ml. In some embodiments, it may be administered at a concentration of about 1 mg/ml to about 5 mg/ml, about 5 mg/ml to about 10 mg/ml, about 10 mg/ml to about 15 mg/ml, about 15 mg/ml to about 20 mg/ml, about 20 mg/ml to about 25 mg/ml, about 25 mg/ml to about 30 mg/ml, about 30 mg/ml to about 35 mg/ml, about 35 mg/ml to about 40 mg/ml, about 40 mg/ml to about 45 mg/ml, about 45 mg/ml to about 50 mg/ml, about 50 mg/ml to about 55 mg/ml, about 55 mg/ml to about 60 mg/ml, about 60 mg/ml to about 65 mg/ml, about 65 mg/ml to about 70 mg/ml, about 70 mg/ml to about 75 mg/ml, about 75 mg/ml to about 80 mg/ml, about 80 mg/ml to about 85 mg/ml, about 85 mg/ml to about 90 mg/ml, about 90 mg/ml to about 95 mg/ml, or about 95 mg/ml to about 0.1 g/ml.

In some embodiments, the OPN variant may be administered at a concentration of about 0.1 g/ml to about 1 g/ml. In other embodiments, it may be administered at a concentration of about 0.1 g/ml to about 0.15 g/ml, about 0.15 g/ml to about 0.2 g/ml, about 0.2 g/ml to about 0.25 g/ml, about 0.25 g/ml to about 0.3 g/ml, about 0.3 g/ml to about 0.35 g/ml, about 0.35 g/ml to about 0.4 g/ml, about 0.4 g/ml to about 0.45 g/ml, about 0.45 g/ml to about 0.5 g/ml, about 0.5 g/ml to about 0.55 g/ml, about 0.55 g/ml to about 0.6 g/ml, about 0.6 g/ml to about 0.65 g/ml, about 0.65 g/ml to about 0.7 g/ml, about 0.7 g/ml to about 0.75 g/ml, about 0.75 g/ml to about 0.8 g/ml, about 0.8 g/ml to about 0.85 g/ml, about 0.85 g/ml to about 0.9 g/ml, about 0.9 g/ml to about 0.95 g/ml, or about 0.95 g/ml to about 1 g/ml.

In certain embodiments, the OPN variant may be administered in a daily dosage of about 0.05 mg/kg of body weight to about 1 mg/kg of body weight. In the context of the present invention, the unit “mg/kg” or “g/kg” mentioned in the context of a daily dosage of the OPN variant, relates to the daily amount of the OPN variant in mg or g per kg body weight of the subject to be treated.

In some embodiments, it may be administered in a daily dosage of about 0.05 mg/kg to about 0.1 mg/kg, about 0.1 mg/kg to about 0.15 mg/kg, about 0.15 mg/kg to about 0.2 mg/kg, about 0.25 mg/kg to about 0.3 mg/kg, about 0.3 mg/kg to about 0.35 mg/kg, about 0.35 mg/kg to about 0.4 mg/kg, about 0.4 mg/kg to about 0.45 mg/kg, about 0.45 mg/kg to about 0.5 mg/kg, about 0.55 mg/kg to about 0.6 mg/kg, about 0.6 mg/kg to about 0.65 mg/kg, about 0.65 mg/kg to about 0.7 mg/kg, about 0.7 mg/kg to about 0.75 mg/kg, about 0.75 mg/kg to about 0.8 mg/kg, about 0.85 mg/kg to about 0.9 mg/kg, about 0.9 mg/kg to about 0.95 mg/kg, or about 0.95 mg/kg to about 1 mg/kg.

In other embodiments, the OPN variant may be administered in a daily dosage of about 1 mg/kg to about 0.1 g/kg of bodyweight. In other additional embodiments, it may be administered in a daily dosage of about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, about 25 mg/kg to about 30 mg/kg, about 30 mg/kg to about 35 mg/kg, about 35 mg/kg to about 40 mg/kg, about 40 mg/kg to about 45 mg/kg, about 45 mg/kg to about 50 mg/kg, about 50 mg/kg to about 55 mg/kg, about 55 mg/kg to about 60 mg/kg, about 60 mg/kg to about 65 mg/kg, about 65 mg/kg to about 70 mg/kg, about 70 mg/kg to about 75 mg/kg, about 75 mg/kg to about 80 mg/kg, about 80 mg/kg to about 85 mg/kg, about 85 mg/kg to about 90 mg/kg, about 90 mg/kg to about 95 mg/kg, or about 95 mg/kg to about 0.1 g/kg.

In some embodiments of the invention, the OPN variant may be administered in a daily dosage of about 0.1 g/kg to about 1 g/kg of bodyweight. In certain embodiments, it may be administered in a daily dosage of about 0.1 g/kg to about 0.15 g/kg, about 0.15 g/kg to about 0.2 g/kg, about 0.2 g/kg to about 0.25 g/kg, about 0.25 g/kg to about 0.3 g/kg, about 0.3 g/kg to about 0.35 g/kg, about 0.35 g/kg to about 0.4 g/kg, about 0.4 g/kg to about 0.45 g/kg, about 0.45 g/kg to about 0.5 g/kg, about 0.5 g/kg to about 0.55 g/kg, about 0.55 g/kg to about 0.6 g/kg, about 0.6 g/kg to about 0.65 g/kg, about 0.65 g/kg to about 0.7 g/kg, about 0.7 g/kg to about 0.75 g/kg, about 0.75 g/kg to about 0.8 g/kg, about 0.8 g/kg to about 0.85 g/kg, about 0.85 g/kg to about 0.9 g/kg, about 0.9 g/kg to about 0.95 g/kg, or about 0.95 g/kg to about 1 g/kg.

In particular embodiments, the OPN variant may be administered in a daily dosage of about 1 g/kg to about 5 g/kg of bodyweight. In some embodiments, it may be administered in a daily dosage of about 1 g/kg to about 1.5 g/kg, about 1.5 g/kg to about 2 g/kg, about 2 g/kg to about 2.5 g/kg, about 2.5 g/kg to about 3 g/kg, about 3 g/kg to about 3.5 g/kg, about 3.5 g/kg to about 4 g/kg, about 4 g/kg to about 4.5 g/kg, about 4.5 g/kg to about 5 g/kg.

In certain embodiments, the OPN variant is administered in a daily dosage in the range of about 0.05 mg/kg to 5 g/kg. For example, the OPN variant may be administered in a daily dosage in the range of about 1 mg/kg to 0.5 g/kg. Alternatively, the OPN variant may be administered in a daily dosage in the range of about 0.005 g/kg to 0.2 g/kg. The OPN variant may e.g. be administered in a daily dosage in the range of about 0.01 g/kg to 0.1 g/kg.

In other embodiments, the OPN variant is administered in a daily dosage in the range of about 1 mg/kg body weight to 300 mg/kg body weight. For example, the OPN variant may be administered in a daily dosage in the range of about 5 mg/kg body weight to 250 mg/kg body weight. Alternatively, the OPN variant may be administered in a daily dosage in the range of about 10 mg/kg body weight to 200 mg/kg body weight. The OPN variant may e.g. be administered in a daily dosage in the range of about 30 mg/kg body weight to 150 mg/kg body weight.

Yet an aspect of the invention relates to the use of an OPN variant comprising, or even consisting of, an OPN variant in the manufacture of a medicament for use in the treatment or prevention of cancer involving at least one cancer tumor.

Yet an aspect of the invention pertains to a method of treating or preventing cancer, the method comprising: administering to a subject having cancer, or to a subject being at risk of getting cancer, an amount of an OPN variant comprising, or even consisting of, an OPN variant effective to treat or prevent said cancer, and where said cancer involves at least one cancer tumor.

In some preferred embodiments of the invention the method comprises administering to a subject having the cancer, or to a subject being at risk of getting the cancer, an amount of an OPN variant comprising, or even consisting of, an OPN variant effective to suppress tumor cell growth or replication.

The method of treatment may for example be a method of reducing the risk of or preventing metastasis in a subject having a cancer involving at least one cancer tumor, for example a cancer tumor having an elevated level of OPN, the method comprising: administering to the subject an amount of an OPN variant effective to reduce the risk of, or even prevent, metastasis.

An aspect of the invention pertains to methods of suppressing tumor cell growth or replication in a subject by administering to the subject the OPN variant, or a pharmaceutical composition comprising the OPN variant in an amount effective to suppress tumor cell growth or replication. Such an effective amount may be referred to herein as a therapeutically effective amount.

The term “effective amount” or “therapeutically effective amount” as used herein means that the amount of the OPN variant administered to the subject directly or comprised within a pharmaceutical composition or nutritional supplement is of sufficient quantity to cause the mentioned effect, for example suppression of tumor growth and/or suppression of tumor cell growth or replication in the subject. The effective amounts may be determined empirically by persons of skill in the art, for example medical practitioners. Factors, such as age, height and weight, of a subject may be considered when for example determining the amount of the OPN variant effective to suppress tumor growth and/or tumor cell growth or replication.

Another aspect of the invention pertains to a pharmaceutical composition comprising:
an OPN variant, and
a pharmaceutically acceptable carrier.

The OPN variant is preferably present in a pharmaceutically effective amount.

In some preferred embodiments of the invention the pharmaceutical composition comprises the OPN variant in an amount in the range of 0.01-90% (w/w). For example, the pharmaceutical composition may comprise the OPN variant in an amount in the range of 0.1-80% (w/w). Alternatively, the pharmaceutical composition may comprise the OPN variant in an amount in the range of 1-70% (w/w).

In some embodiments of the invention the pharmaceutical composition comprises the OPN variant in an amount in the range of 5-60% (w/w). For example, the pharmaceutical composition may comprise the OPN variant in an amount in the range of 10-50% (w/w). Alternatively, the pharmaceutical composition may comprise the OPN variant in an amount in the range of 0.1-20% (w/w).

In addition to the OPN variant, the pharmaceutical composition may furthermore comprise one or more additional therapeutic agent(s). The one or more additional therapeutic agent(s) is preferably an anti-cancer agent.

Examples of targeted therapeutic agents include small molecules, such as imatinib mesylate (GLEEVEC®, also known as STI-571), Gefitinib (IRESSA®, also known as ZD1839), Erlotinib (Tarceva®), bortezomib (VELCADE®), Bcl-2 inhibitors (for example obatoclax mesylate, ABT-263, and gossypol), PARP inhibitors (for example iniparib, olaparib), Janus kinase inhibitors, PI3K inhibitors, Apatinib (a selective VEGF receptor 2 inhibitor), and salinomycin. Examples of targeted therapeutic agents also include monoclonal antibodies, such as Rituximab (marketed as MABTHERA® or RITUXAN®), Trastuzumab (HERCEPTIN®), Cetuximab (ERBITUX®), Bevacizumab (AVASTATIN®). Examples also include antibody-drug conjugates.

Examples of chemotherapeutic agents include alkylating agents (for example cisplatin, carboplatin, oxaliplatin, mechlorethamine, cyclophosphamide, chlorambucil, ifosfamide), anti-metabolites (for example purines (such as azathioprine, mercaptopurine) and pyrimidines), plant alkaloids and terpenoids (for example vinca alkaloids such as vincristine, vinblastine, vinorelbine, and vindesine, podophyllotoxins, and taxanes), topoisomerase inhibitors (for example irinotecan, topotecan, amsacrine, etoposide, etoposide phosphate, and teniposide), and cytotoxic inhibitors (for example actinomycin such as actinomycin D, anthracyclines such as doxorubicin (L01DB01), daunorubicin (L01DB02), valrubicin, idarubicin, epirubicin (L01DB03), and other cytotoxic antibiotics such as bleomycin (L01DC01), plicamycin (L01DC02), and mitomycin (L01DC03)).

Another example of a useful therapeutic agent is an integrin blocking agent, e.g. an alphaᵥ beta₃ integrin blocking agent such as cilengitide.

Examples of immunosuppressive agents include glucocorticoids, cytostatics (for example alkylating agents and antimetabolites such as folic acid analogues (for example methotrexate), purine analogues (for example azathioprine, mercaptopurine), pyrimidine analogues, and protein synthesis inhibitors), antibodies (for example polyclonal and monoclonal), drugs acting on immunophilins (for example ciclosporin, tacrolimus, sirolimus), and other drugs, including interferons, opioids, TNF binding proteins, mycophenolate, and small biological agents (for example fingolimod, myriocin).

Other aspects described herein include pharmaceutical compositions comprising the OPN variant and a pharmaceutically acceptable carrier, one or more compatible solid or liquid fillers, or one or more diluents or encapsulating agents appropriate for the administration to a human or other animal. A carrier (or other agents) should be sufficiently pure and sufficiently low-toxic in order to be regarded as appropriate for the administration to a subject being treated. A carrier may be inert or may have its own pharmaceutically favorable properties. An amount of the carrier used in combination with the OPN variant may be sufficient to improve delivery and effectiveness of the OPN variant (for example the OPN variant delivery to cells or OPN variant uptake by cells) and may be determined empirically by those of skill in the art.

Examples of additional carriers or other (inactive) agents that may be comprised within the pharmaceutical compositions described herein include sugars, such as lactose, glucose and saccharose; starches, such as corn starch and potato starch; cellulose and derivatives thereof, such as sodium carboxymethylcellulose, ethylcellulose and methylcellulose; powdered tragacanth gum; gelatine; talc; solid lubricants, such as stearinic acid and magnesium stearate; calcium sulfate, vegetable oils, such as peanut butter, cottonseed oil and corn oil; polyols, such as propylene glycol, glycerin, sorbitol, mannitol and polyethylene glycol; alginic acid; emulsifiers, such as TWEEN®; wetting agents, such as sodium laurylsulfate; dyes; correctives; pelletizing agents; stabilizers; antioxidants; preservatives; pyrogen-free water; isotonic physiological solution, glucose solution and phosphate-buffered solutions; sweeteners, such as glycerine, propylene glycol, sorbitol, saccharose); correctives; flavoring agents; dyes; and preservatives, such as methyl- or n-propyl-p-hydroxy benzoate, sorbic acid, methyl paraben, benzoate.

Optional active agents which do not significantly affect the activity of the compound of the present invention may be added to the pharmaceutical composition. Such active agents include anticancer targeted therapeutics and chemotherapeutic agents, and immunosuppressive or immunostimulating agents.

In certain embodiments, the pharmaceutical compositions containing the OPN variant are formulated for mucosal and/or oral administration. The compositions may be administered orally, sublingually, or buccally in standard dosage forms containing conventional nontoxic pharmaceutically acceptable carriers, adjuvants and media. The terms “oral” or “orally” may encompass “sublingual” or “sublingually” or “buccal” or “buccally”.

Pharmaceutically acceptable carriers specific for mucosal and/or oral administration are well-known in the art and include one or more sugars, starches, cellulose and derivative thereof, malt, gelatin, talc, calcium sulfate, vegetable oils, synthetic oils, polyols, alginic acid, phosphate-buffered solutions, emulsifiers, isotonic physiological solutions, ethanol, glycerin, propylene glycol, polyethylene glycol, sugar solution, sorbitol and water. Such compositions may also contain a demulcent.

Forms suitable for oral administration include tablets or granules, hard or soft capsules, pastilles, troches, suspensions in water or oil, emulsions, dispersible powders or granules, or syrups or elixirs. Pharmaceutical compositions formulated for oral administration may be prepared according to any method known in the art for the preparation of such compositions.

Tablets typically contain conventional pharmaceutically compatible auxiliary agents, such as inert diluents, such as calcium carbonate, sodium carbonate, mannitol, lactose and cellulose; binders, such as starch, gelatin and saccharose; dispersing agents, such as starch, alginic acid and croscarmellose; lubricants, such as magnesium stearate, stearic acid and talc. Glidants, such as silicon dioxide, may be used to improve fluidity characteristics of a powder composition. For appearance, dyes such as FD&C dyes may be added. Sweeteners and correctives, such as aspartame, saccharine, menthol, peppermint and fruit flavors are useful as adjuvants for chewable tablets. Capsules (including sustained release and delayed release preparations) typically contain one or more solid diluents described above. The selection of carrier components often depends on secondary factors, such as flavor, price and storage stability.

Pharmaceutical compositions in the form of tablets or capsules may also be coated using the conventional methods, typically with a pH-dependent coating. Such dosage forms typically comprise one or more components from among cellulose acetate phthalate, polyvinyl acetate phthalate, hydroxypropylmethylcellulose phthalate, ethyl cellulose, Eudragit coatings, waxes and shellac and other materials.

Preparations containing the OPN variant for oral administration may be formulated into hard gelatin capsules, wherein the OPN variant is mixed with an inert solid diluent, for example calcium carbonate, calcium phosphate and kaolin, or in the form of soft gelatin capsules, wherein the OPN variant is mixed with water or an oil medium, for example peanut butter, liquid paraffin or olive oil.

Aqueous suspensions may comprise the OPN variant in a mixture with excipients suitable for obtaining aqueous suspensions. These excipients may be suspending agents, for example, carboxymethylcellulose sodium, methylcellulose, hydropropylmethyl cellulose, sodium alginate, polyvinyl pyrrolidone, tragacanth gum and Arabic gum; dispersing or wetting agents; naturally-occurring phosphatides, for example, lecithin, or products of condensation of alkylenoxide with fatty acids, for example, polyoxyethylene stearate, or products of condensation of ethylene oxide with long-chain aliphatic alcohols, for example, with heptadecaethylene oxycetanol, or products of condensation of ethylene oxide with partial esters produced from fatty acids and hexitol, such as substituted polyoxyethylene sorbitol, or products of condensation of ethylene oxide with partial esters produced from fatty acids and hexitol anhydrides, for example, substituted polyoxyethylene sorbitan. Aqueous suspensions may also contain some preservatives, for example ethyl- or n-propyl-p-hydroxybenzoate.

Oil suspensions may be prepared by suspending the OPN variant in a vegetable oil, for example, peanut butter, olive oil, sesame oil and coconut oil, or in a mineral oil, such as liquid paraffin. Oil suspensions may contain a thickening agent, for example, bee wax, solid paraffin or cetyl alcohol. Some sweeteners, such as mentioned above, and correctives can be added to obtain pleasant oral preparations. These compositions may be preserved by adding an anti-oxidant, such as ascorbic acid.

When the OPN variant exhibits insufficient solubility, solubilization methods may be used. Such methods are known to ones skilled in this field of art and comprise the use of co-solvents, such as dimethylsulfoxide (DMSO), the use of surfactants, such as TWEEN®, or dissolving in an aqueous solution of sodium bicarbonate and other methods.

The pharmaceutical compositions described herein may also be in the form of oil-in-water emulsions. The oil phase may represent a vegetable oil, for example, olive oil or peanut butter, or a mineral oil, for example, liquid paraffin or mixtures thereof. The appropriate emulsifiers may be naturally occurring gums, for example, Arabic gum or tragacanth gum, naturally occurring phosphatides, for example, soybean lecithin, and esters or partial esters produced from fatty acids and hexitol anhydrides, for example, sorbitan monooleate, and products of condensation of said partial esters with ethylene oxide, for example, polyoxyethylene sorbitan monooleate.

Dispersing powders and granules suitable for preparing an aqueous suspension include the OPN variant in a mixture with a dispersing or wetting agent, suspending agent and one or more preservatives. The appropriate dispersing or wetting agents and suspending agents include agents already exemplified above.

In one embodiment, the OPN variant or pharmaceutical compositions comprising the OPN variant may be administered in a nasal dosage form (for example nasal spray). Such compositions typically contain one or more fillers, such as saccharose, sorbitol and mannitol, and binders, such as Arabic gum, microcrystal cellulose, carboxymethylcellulose and hydroxypropylmethylcellulose. Glidants, lubricants, sweeteners, dyes, antioxidants and correctives described above can also be incorporated.

Pharmaceutical compositions for inhalation may be formulated in a solution, suspension or emulsion, which may be administered in the form of a dry powder or in the form of an aerosol using a conventional propellant (for example, dichlorodifluoromethane and trichlorofluoromethane).

In some preferred embodiments of the invention, the pharmaceutical composition is formulated for oral, sublingual, buccal, or nasal administration.

In other embodiments of the invention, the pharmaceutical composition is formulated for intravenous administration, e.g. for injection.

In some embodiments of the invention, the pharmaceutical composition is in a dosage form, which contains 90%-110% (w/w) of the daily dosage for an adult subject. In other embodiments of the invention, the pharmaceutical composition is in a dosage form, which contains 45%-55% (w/w) of the daily dosage for an adult subject. In further embodiments of the invention, the pharmaceutical composition is in a dosage form, which contains 28%-38% (w/w) of the daily dosage for an adult subject.

In some embodiments of the invention, the pharmaceutical composition is in a dosage form, which dosage form contains the OPN variant in an amount in the range of 0.1 mg-10 g per dosage form. For example, the oral dosage form may contain an amount of the OPN variant in the range of 1 mg-1 g per dosage form. Alternatively, the oral dosage form may contain an amount of the OPN variant in the range of 10 mg-800 mg per dosage form. The oral dosage form may e.g. contain an amount of the OPN variant in the range of 25 mg-500 mg per dosage form.

Yet an aspect of the invention pertains to a nutritional supplement comprising
a nutritionally effective amount of an OPN variant, and
one or more components selected from the group consisting of a carbohydrate source, a lipid source, and a protein source.

In some preferred embodiments of the invention the nutritional supplement comprises the OPN variant in an amount in the range of 0.01-90% (w/w). For example, the nutritional supplement may comprise the OPN variant in an amount in the range of 0.1-80% (w/w). Alternatively, the nutritional supplement may comprise the OPN variant in an amount in the range of 1-70% (w/w).

In some embodiments of the invention the nutritional supplement comprises the OPN variant in an amount in the range of 5-60% (w/w). For example, the nutritional supplement may comprise the OPN variant in an amount in the range of 10-50% (w/w). Alternatively, the nutritional supplement may comprise the OPN variant in an amount in the range of 0.1-20% (w/w).

In other embodiments of the invention the nutritional supplement comprises the OPN variant in an amount in the range of 0.001-5% (w/w). For example, the nutritional supplement may comprise the OPN variant in an amount in the range of 0.005-2% (w/w). Alternatively, the nutritional supplement may comprise the OPN variant in an amount in the range of 0.01-1% (w/w). The nutritional supplement may e.g. comprise the OPN variant in an amount in the range of 0.05-0.5% (w/w).

Nutritional supplements comprising the OPN variant can be pre-packaged in liquid or powdered form (for example canned or bottled liquid drink). In some embodiments, the powdered form is added to a food or beverage to provide additional nutrients. In certain embodiments, the nutritional beverages are formulated with, for example, fruit, vegetables, yogurt, milk, and/or ice cream. In some embodiments, the nutritional supplements are blended to a smoothie consistency. In particular embodiments, the nutritional beverages are fortified with, for example, protein, vitamins, minerals, antioxidants, probiotics, and/or prebiotics. In certain embodiments, the nutritional beverages are lactose-free and/or gluten-free. In some embodiments, the nutritional supplements are organic. Examples of pediatric nutritional beverages include PEDIASURE®, PEDIASMART®, and RESOURCE® Just For Kids. Examples of adult nutritional beverages include ENSURE®, BOOST®, NESTLE® CARNATION® INSTANT BREAKFAST®, GLUCERNA®, GLYTROL®, NUTREN®, and PEPTAMEN®. Nutritional supplements also include milk, both soymilk and cow's milk (for example whole, semi-skim or low-fat, skim or non-fat (for example Cravendale), lactose-free (for example LACTOFREE®)).

The amount of the OPN variant comprised within the nutritional supplements described herein may be the same or similar to those amounts described above for the pharmaceutical compositions comprising the OPN variant, but may be lesser or greater amounts also. In particular embodiments, the amount of the OPN variant in the nutritional supplement is about 0.05 mg/ml to about 1 g/ml. In some embodiments, the subject may be a mammal. In some embodiment the subject may be murine, canine, feline, ovine, bovine, porcine, or equine, while in other embodiments the subject is human.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Each of the foregoing patents, patent applications and references is hereby incorporated by reference, particularly for the teaching referenced herein.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### EXAMPLES

### Example 1

### Oral Administration of Osteopontin to 129B6F1 Tumor-Bearing Mice

A bovine OPN preparation (Lacprodan OPN-10®, Aria Foods Ingredients, Viby, Denmark) was dissolved in deionized water, filtered, and administered at final concentrations of 0.03, 0.12, or 0.3 mg/ml to 129B6F1 tumor-bearing mice starting either on the day of tumor cell inoculation (Day 0) or five days later (Day 5). The mice were initially inoculated with 5×10⁴ mycoplasma-free 275-3-2 murine ras-transformed fibroblast cells (see Wu 2000). Tumor size was measured with calipers and monitored until the tumors reached 20% of the body weight of the animals, at which time the mice were sacrificed and tissue/plasma samples collected. Statistical significance was calculated using one-way ANOVA with Bonferroni post-test.

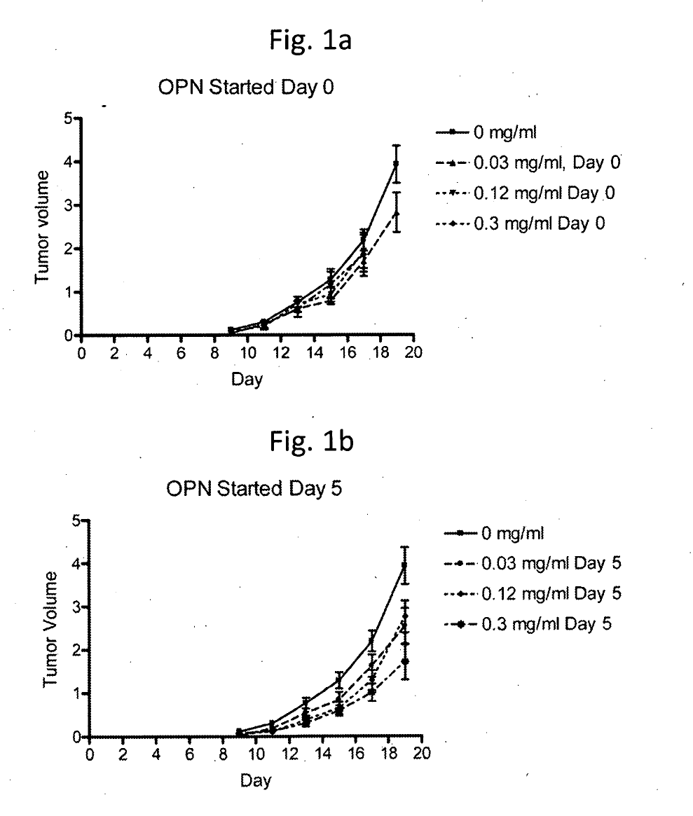

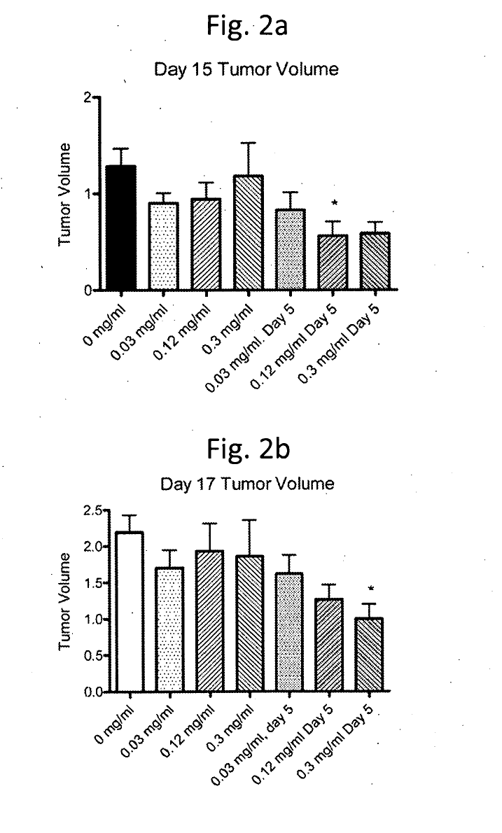

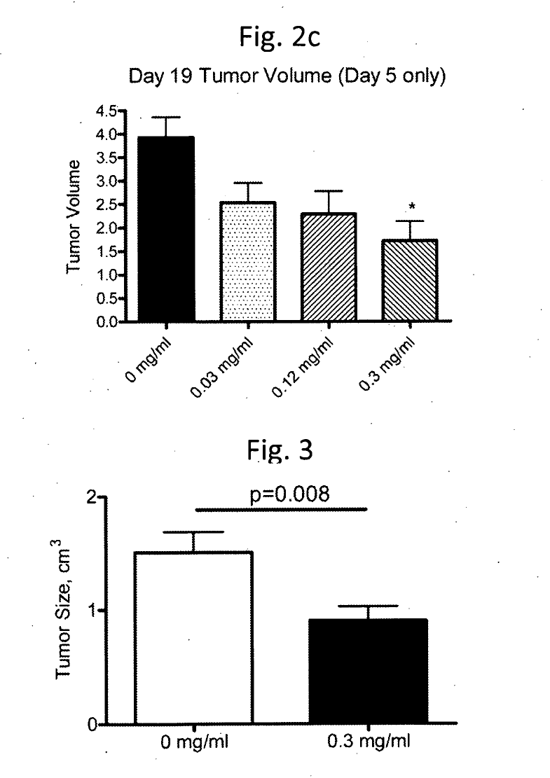

Tumors were initially detected at day 9. Starting on day 19, some tumors had grown so large that the mice had to be sacrificed. The experiment was terminated on day 25. FIG. 1A shows that administration of the OPN preparation at the time of tumor injection had no significant effect on the size of the tumors up to 17 days. When the administration of the OPN preparation was initiated five days after tumor cell injection, however, there was a statistically significant decrease in the size of the tumor in both the 0.12 mg/ml and 0.3 mg/ml groups, at 15 and 17 days, respectively (FIG. 1b). FIG. 2 shows a comparison of tumor sizes in all the groups on days 15, 17 and 19; significant differences are indicated. FIG. 3 shows the mean tumor size of control and OPN-fed mice, combined results from three independent experiments. N=30 (0 mg/ml OPN preparation) or 32 (0.3 mg/ml OPN preparation).

These results clearly demonstrate that bovine OPN preparation, administered orally, can suppress the rate of growth of cancer tumors and possibly even prevent tumor growth. The amount of protein administered to the mice, about 1.5 mg, does not significantly alter their total protein intake, nor is there any effect of this treatment on mouse weight. Therefore, we conclude that this treatment has a specific effect on tumor or associated host cells that slows the growth of the tumor. Dosing experiments suggest that the highest dose of OPN preparation used, 0.3 mg/ml is most effective, and even higher doses may have larger effects on tumor growth.

### Example 2

### Effect of Orally Administered OPN on Primary Tumor Growth and Metastasis in a Mouse Model of Breast Cancer

4T1 cells are transformed mouse mammary epithelial cells (Aslakson 1992) that form metastatic tumors after orthotopic injection into the mouse mammary gland (Lelekakis 1999). This spontaneous metastasis formation—primarily to the lungs although other tissues can be involved—is a key feature of these cells that more faithfully reflects the development of metastases in human cancers than direct injection metastasis models. Accordingly, these cells are widely used as a model for aggressive human breast cancer: 4T1 cells also express high levels of OPN (Mi 2004), which is required for maximal tumor growth.

A. Mouse tumor development. 4T1 cells will be obtained from ATCC. In initial experiments, expression of OPN will be confirmed and cells will be tested for mycoplasma and remediated if necessary. Cells will be harvested while in exponential growth, washed, and 1×10⁵ cells will be injected into the mammary fat pad of 36 syngeneic Balb/c mice. The mice will be randomized into three groups, two of which will receive 0.3 mg/ml OPN preparation in drinking water starting five days after tumor cell injection. The 4T1 cells form tumors rapidly: tumors are expected to be detected after 7-10 days, with maximal tumor volume reached by 25-30 days. Control mice and one group of OPN-fed mice (n=12 per group) will be sacrificed when any one mammary tumor reaches 20% of body weight, or if any pathology is detected. If, as we expect, the growth of the primary tumors will be suppressed in the OPN-fed animals, the second group of OPN-fed mice will be maintained until their tumors reach 20% of body weight. At sacrifice, a necropsy will be performed and sites of metastasis will be noted. Blood, tumors, lungs, and other metastatic tissues will be collected.

B. Analysis of mouse tissues. Primary tumors will be divided into three crosswise sections for cryopreservation, formaldehyde fixation and biochemical analyses. Lungs will be fixed for determination of metastatic burden, which will be assessed by counting surface nodules, and by histological examination of several lung sections. The number of surface metastases per mouse will be compared between mice which have been subjected to oral administration of OPN and control mice as the primary outcome. If differences in primary tumor growth rate are seen, blood vessel density and vessel morphology in different tumors will be assessed by CD31 and vWF staining, and lymphatic vessel density by staining for LYVE-1. If time allows, aspects of VEGF signaling will be analyzed: expression of various VEGF isoforms, expression of VEGF receptors, and the level of phosphorylation of those receptors will be assessed by western blot to test the hypothesis that oral administration of OPN causes altered VEGF signaling. These analyses will be carried out in both primary tumors and in metastatic lesions.

C. Expected results. Our hypotheses and previous data suggest that oral administration of OPN variants will suppress the growth of the primary mammary tumors in mice injected with 4T1 cells. If this is the case, and the corresponding metastatic burden is lower in OPN-fed mice, an additional group of OPN-fed mice will be included, in which tumors will be allowed to grow to the same size as control tumors, when they will be sacrificed and the metastatic burden determined. If lower numbers of metastases are seen in these animals as compared to controls, we will conclude that oral administration of OPN has a direct effect on metastasis. We anticipate that oral administration of OPN variants may result in increased blood vessel size, accompanied by increased activity of the VEGF signaling pathway.

### Example 3

### Determination of the Level OPN in a Tumor

This example describes how the level of OPN in a tumor is determined.

### Sample Preparation:

A sample of the tumor in question is obtained and subsequently flash frozen and homogenized (about 50 mg tumor sample/0.25 ml buffer in Cell Lysis Buffer (Cell Signaling Technologies, Cat #9803) in the presence of protease inhibitors (Roche Applied Science cat #05 892 791 001). Homogenization is performed in 1.8 ml eppendorf tubes using plastic pestles on ice, for 30 secs to 1 minute.

### Determination of Total Protein:

The protein concentration of the tumor extract is determined using a bicinchoninic acid (BCA) assay kit (Pierce Biotechnology Cat #23227).

### Determination of the OPN Level:

The OPN level the tissue extract is determined by ELISA using antibodies raised against the recombinant OPN which is native to the subject from which the blood sample is taken.

In the case of human subjects, monoclonal antibodies against human serum OPN should be used. One may for example use the Assay Designs Kit (Enzo Life. Sciences, Cat# ADI-900-142) according to the manufacturer's instructions. The assay is calibrated with controlled samples of purified recombinant human OPN in varying concentrations from 2-32 ng/ml.

In the case of murine subjects, monoclonal antibodies against murine serum OPN should be used. One may for example use the Mouse Osteopontin ELISA Duoset Kit (R&D Systems, Cat # DY441) according to the manufacturer's instructions. The assay is calibrated with controlled samples of purified recombinant murine serum OPN in varying concentrations from 31-1000 pg/ml.

The resulting level of OPN in the tumor is presented as nanogram OPN per microgram total protein in the tumor sample.

### Example 4

### Determination of the Concentration of OPN in Plasma

This example describes how the concentration of OPN in plasma is determined.

### Sample Preparation:

Plasma is prepared from blood collected from the subject in the presence of 1.8 mg NaEDTA per mL blood.

### Determination of the OPN Concentration:

The concentration of OPN is determined by ELISA using monoclonal antibodies raised against recombinant OPN which is native to the subject from which the blood sample is taken.

In the case of human subjects, monoclonal antibodies against human serum OPN should be used. One may for example use the Assay Designs Kit (Enzo Life Sciences, Cat# ADI-900-142) according to the manufacturer's instructions. The level of OPN in normal human plasma ranges from 14-45 ng/ml. The assay is calibrated with controlled samples of purified recombinant human OPN in varying concentrations from 2-32 ng/ml.

In the case of murine subjects, monoclonal antibodies against murine serum OPN should be used. One may for example use the Mouse Osteopontin ELISA Duoset Kit (R&D Systems, Cat # DY441) according to the manufacturer's instructions. The assay is calibrated with controlled samples of purified recombinant murine serum OPN in varying concentrations from 31-1000 pg/ml.

The resulting level of OPN in the plasma is presented as nanograms OPN per mL plasma.

### REFERENCES

| | |
|---|---|
| Aslakson 1992 | Aslakson, C. J., and Miller, F. R. (1992). Selective events in |
| | the metastatic process defined by analysis of the sequential |
| | dissemination of subpopulations of a mouse mammary tumor. |
| | Cancer Res 52, 1399-1405. |
| Anborgh 2009 | Anborgh, P. H., Wilson, S. M., Tuck, A. B., Winquist, E., |
| | Schmidt, N., Hart, R., Kon, S., Maeda, M., Uede, T., Stitt, L. W., |
| | et al. (2009). New dual monoclonal ELISA for measuring |
| | plasma osteopontin as a biomarker associated with survival |
| | in prostate cancer: clinical validation and comparison of |
| | multiple ELISAs. Clin Chem 55, 895-903. |
| Butler 1996 | Butler W T, et al. 1996. Osteopontin. In Bilezekian J P, Rai L G, |
| | Rodan G A (eds.) Principles of bone biology. Academic |
| | Press, San Diego, Calif., U.S.A., pp. 167-181. |
| Denhardt 1995 | Denhardt D T, et al. (eds.) 1995. Osteopontin: role in cell |
| | signaling and adhesion. Ann. N. Y. Acad. Sci., 760 |
| Feng 2000 | Feng, F., and Rittling, S. R. (2000). Mammary tumor development |
| | in MMTV-c-myc/MMTV-v-Ha-ras transgenic mice is |
| | unaffected by osteopontin deficiency. Breast Cancer |
| | Res. Treat. 63, 71-79. |
| Franzen 1985 | Franzen A, Heineg 1985. Biochem. J. 232: 715-724 |
| Lelekakis 1999 | Lelekakis, M., Moseley, J. M., Martin, T. J., Hards, D., Williams, E., |
| | Ho, P., Lowen, D., Javni, J., Miller, F. R., Slavin, J., |
| | et al. (1999). A novel orthotopic model of breast cancer metastasis |
| | to bone. ClinExpMetastasis 17, 163-170. |
| Mi 2004, | Mi, Z., Guo, H., Wai, P. Y., Gao, C., Wei, J., and Kuo, P. C. |
| | (2004). Differential osteopontin expression in phenotypically |
| | distinct subclones of murine breast cancer cells mediates |
| | metastatic behavior. J Biol Chem 279, 46659-46667. |
| Nelson 2005 | Nelson et al, Annals of Internal Medicine, Volume 143, Number |
| | 5, 6 September 2005, p. 362-379 |
| Rittling 1997 | Rittling, S. R., and Novick, K. E. (1997). Osteopontin expression |
| | in mammary gland development and tumorigenesis. Cell |
| | Growth and Differentiation 8, 1061-1069. |
| Rittling 2002 | Rittling, S. R., Chen, Y., Feng, F., and Wu, Y. (2002). Tumor- |
| | derived osteopontin is soluble, not matrix associated. Journal |
| | of Biological Chemistry 277, 9175-9182. |
| Senger 1988 | Senger D R, et al. 1988. Cancer Res. 48: 5770-5774 |
| Sorensen 1994 | Sorensen E S, et al. 1994. Protein Sci. 4: 2040-2049 |
| Tomayko 1989 | Tomayko and Reynolds; Cancer Chemotherapy and Pharmacology |
| | (1989), Vol. 24, No. 3, p. 148-154 |
| Tuck 1998 | Tuck, A. B., O'Malley, F. P., Singhal, H., Harris, J. F., Tonkin, K. S., |
| | Kerkvliet, N., Saad, Z.; Doig, G. S., and Chambers, A. F. |
| | (1998). Osteopontin expression in a group of lymph node |
| | negative breast cancer patients. International Journal of |
| | Cancer 79, 502-508. |
| Wu 2000 | Wu, Y., et al., Br. J. Cancer (2000) 83: 156-163 |

1. An OPN variant for use in the treatment or prevention of cancer involving at least one cancer tumor,
wherein said OPN variant comprises an active OPN molecule having a sequence identity of at least 80% relative to SEQ ID NO. 1 or SEQ ID NO. 2, and/or an active fragment of an OPN molecule, said fragment having a sequence identity of at least 80% relative to the sequence of position 17-163 of SEQ ID NO. 1 or to the sequence of position 17-170 of SEQ ID NO. 2.

2. The OPN variant according to claim 1, wherein the cancer tumor has an elevated level of OPN.

3. The OPN variant according to claim 1 or 2, wherein the treatment or prevention is by oral administration.

4. The OPN variant according to any of the preceding claims, wherein the treatment or prevention is by parenteral administration, such as e.g. by intravenous or intraperitoneal administration.

5. The OPN variant according to any of the preceding claims, for suppressing tumor cell growth or replication.

6. The OPN variant according to any of the preceding claims, for suppressing tumor growth.

7. The OPN variant according the any of the preceding claims, for preventing tumor cell growth or replication.

8. The OPN variant according to any of the preceding claims, for preventing tumor growth.

9. The OPN variant according to any of the preceding claims, for reducing the risk of metastasis in a subject having a cancer involving at least one cancer tumor.

10. The OPN variant according to any of the preceding claims, for preventing metastasis in a subject having a cancer involving at least one cancer tumor.

11. The OPN variant according to any of the preceding claims, wherein the subject to be treated has a cancer involving at least one cancer tumor having an elevated level of OPN.

12. The OPN variant according to any of the preceding claims, wherein the subject having the cancer tumor has an elevated concentration of OPN in its plasma.

13. OPN variant according to any of the preceding claims, wherein the subject to which the OPN variant is administered has an increased risk of developing a cancer involving at least one cancer tumor.

14. The OPN variant according to any of the preceding claims, wherein the OPN variant is administered in a daily dosage in the range of about 0.05 mg/kg of body weight to about 5 g/kg of body weight of the subject treated.

15. The OPN variant according to any of the preceding claims, comprising a total amount of active fragments of OPN molecules of at least 10% (w/w) relative to the total weight of the OPN variant.

16. The OPN variant according to any of the preceding claims, comprising a total amount of active OPN molecules in the range of 10-90% (w/w) relative to the total weight of the OPN variant, and a total amount of active fragments of OPN molecules in the range of 10-90% (w/w) relative to the total weight of the OPN variant.

17. The OPN variant according to any of the preceding claims, comprising a total amount of active OPN molecules in the range of 10-40% (w/w) relative to the total weight of the OPN variant, and a total amount of active fragments of OPN molecules in the range of 60-90% (w/w) relative to the total weight of the OPN variant.

18. The OPN variant according to any of the preceding claims, comprising a total amount of active OPN molecules of at least 10% (w/w) relative to the total weight of the OPN variant.

19. The OPN variant according to any of the preceding claims, wherein the OPN variant is isolated from bovine milk.

20. The OPN variant according to any of the preceding claims, wherein said OPN variant is an active OPN molecule having a sequence identity of at least 80% relative to SEQ ID NO. 1, and/or an active fragment of an OPN molecule having a sequence identity of at least 80% relative to the sequence of position 17-163 of SEQ ID NO. 1.

21. The OPN variant according to any of the preceding claims, wherein said OPN variant is an active OPN molecule having a sequence identity of at least 80% relative to SEQ ID NO. 2, and/or an active fragment of an OPN molecule having a sequence identity of at least 80% relative to the sequence of position 17-170 of SEQ ID NO. 2.

22. A method of treating or preventing cancer, the method comprising: administering to a subject having cancer, or to a subject being at risk of getting cancer, an amount of an OPN variant effective to treat or prevent said cancer, and wherein said cancer involves at least one cancer tumor,
said OPN variant comprises an active OPN molecule having a sequence identity of at least 80% relative to SEQ ID NO. 1 or SEQ ID NO. 2, and/or an active fragment of an OPN molecule, said fragment having a sequence identity of at least 80% relative to the sequence of position 17-163 of SEQ ID NO. 1 or to the sequence of position 17-170 of SEQ ID NO. 2.

23. A method of treating or preventing cancer according to claim 22, the method comprising: administering to a subject having the cancer, or to a subject being at risk of getting the cancer, an amount of an OPN variant effective to suppress tumor cell growth or replication, wherein said OPN variant comprises an active OPN molecule having a sequence identity of at least 80% relative to SEQ ID NO. 1 or SEQ ID NO. 2, and/or an active fragment of an OPN molecule, said fragment having a sequence identity of at least 80% relative to the sequence of position 17-163 of SEQ ID NO. 1 or to the sequence of position 17-170 of SEQ ID NO. 2.

23. A method of reducing the risk of or preventing metastasis in a subject having a cancer involving at least one cancer tumor, the method comprising: administering to the subject an amount of an OPN variant effective to reduce the risk of or prevent metastasis,
wherein said OPN variant comprises an active OPN molecule having a sequence identity of at least 80% relative to SEQ ID NO. 1 or SEQ ID NO. 2, and/or an active fragment of an OPN molecule, said fragment having a sequence identity of at least 80% relative to the sequence of position 17-163 of SEQ ID NO. 1 or to the sequence of position 17-170 of SEQ ID NO. 2.

24. A pharmaceutical composition comprising:
an OPN variant comprising an active OPN molecule having a sequence identity of at least 80% relative to SEQ ID NO. 1 or SEQ ID NO. 2, and/or an active fragment of an OPN molecule, said fragment having a sequence identity of at least 80% relative to the sequence of position 17-163 of SEQ ID NO. 1 or to the sequence of position 17-170 of SEQ ID NO. 2, and
a pharmaceutically acceptable carrier.

25. The pharmaceutical composition according to claim 24, comprising the OPN variant in an amount in the range of 0.01-90% (w/w).

26. The pharmaceutical composition according to claim 24 or 25, furthermore comprising one or more additional therapeutic agent(s).

27. The pharmaceutical composition according to claim 26, wherein the one or more additional therapeutic agent(s) comprises an anti-cancer agent.

28. The pharmaceutical composition according to any of the claims 24-27, wherein the pharmaceutical composition is formulated for oral, sublingual, buccal, nasal, or intravenous administration.

29. A nutritional supplement comprising
a nutritionally effective amount of an OPN variant comprising an active OPN molecule having a sequence identity of at least 80% relative to SEQ ID NO. 1 or SEQ ID NO. 2, and/or an active fragment of an OPN molecule, said fragment having a sequence identity of at least 80% relative to the sequence of position 17-163 of SEQ ID NO. 1 or to the sequence of position 17-170 of SEQ ID NO. 2, and
one or more components selected from the group consisting of a carbohydrate source, a lipid source, and a protein source.
